(19) United States
(12) Patent Application Publication
(10) Pub. No.: US 2006/0229355 A1
(43) Pub. Date: Oct. 12, 2006

(54) 3.3'-Diindolylmethane compositions inhibit angiogenesis

(75) Inventors: Leonard F. Bjeldanes, Berkeley, CA (US); Xiaofei Chang, Berkeley, CA (US); Gary L. Firestone, Berkeley, CA (US)

Correspondence Address: RICHARD ARON OSMAN;SCIENCE AND TECHNOLOGY LAW GROUP, 242 AVE VISTA DEL OCEANO, SAN CLEMEMTE, CA, 92672, US

(73) Assignee: The Regents of the University of California

(21) Appl. No.: 11/102,336

(22) Filed: Apr. 8, 2005

(51) Int. Cl. A61K 31/405

(52) U.S. Cl. 514/414

(57) ABSTRACT

The invention provides antiangiogenic compositions and methods of use. The general methods deliver an antiangiogen to a patient determined to be in need thereof, comprising the steps of: (a) administering to the patient a predetermined amount of an antiangiogenic, optionally substituted DIM; and (b) detecting in the patient a resultant antiangiogenic response.

This work was supported by National Institute of Health Grant CA69056, US Army Grant RP950844, and Department of Defense, Army Breast Cancer Research Program Grant DAMDI 7-96-1-6159. The U.S. government may have rights in any patent issuing on this application.

### INTRODUCTION

1. Field of the Invention

The field of the invention is anti-angiogenic 3,3′-diindolylmethane compositions.

2. Background of the Invention

Angiogenesis, the development of new capillaries from an existing vascular network, is an important event in normal and pathological development. Angiogenesis can occur under normal physiological conditions, including embryonic development, female reproductive cycling, and wound healing. However, it is also associated with a large number of diseases, including cancer, cardiovascular diseases, rheumatoid arthritis, psoriasis and diabetic retinopathy. [1; 2]. Successful tumor establishment depends on the angiogenesis process to provide oxygen and nutrients to rapidly proliferating cells [2; 3]. Formation of new blood vessels during this process is critically dependent on the ability of endothelial cells to proliferate, degrade extracellular matrix, migrate and differentiate [4; 5]. At the beginning of tumorigenesis, the tumor cell mass is not vascularized and it does not grow beyond a few cubic millimeters unless vascularization has occurred [3; 6]. In addition, the appearance of a vascular stage in the natural history of a tumor is associated with development of metastases. Therefore, control of tumor angiogenesis is a major area of exploration for the development of cancer preventive and therapeutic agents.

Results of epidemiologic studies and laboratory investigations with rodents and cultured tumor cells provide evidence that phytochemicals in broccoli and other cruciferous vegetables have anticarcinogenic properties [7]. One of these phytochemicals, indole-3-carbinol (I3C), self-condenses at the low pH of stomach to multiple products [8]. 3,3′-Diindolylmethane (DIM) is a major acid-catalyzed product of I3C [9]. Our prior work indicates that DIM can be used as an androgen antagonist to target androgen-dependent tumors (e.g. Le et al., J Biol. Chem. 2003 Jun. 6; 278(23):21136-45; U.S. Ser. No. 10/664,991), and prior reports describing anti-cancer properties of I3C and DIM are directed to cancers which are, or are proposed to be estrogen- or androgen-regulated. These include studies of prostate and mammary cancers (e.g. Chen et al, [10]), and also papillomavirus-induced tumors, including tumors of the larynx and cervix wherein the pathogenesis is said to be related to estrogen metabolism (Newfield et al., Anticancer Res. 1993 Mar.-Apr.;13(2):337-41; Abramson et al., Anticancer Res. 1998 Nov.-Dec.; 18(6B):4569-73; Wiatrak, Curr. Opin. Otolaryngol. Head Neck Surg., 11: 433-41, 2003). We also recently reported that DIM can be used as an immune response activator, with applications to infection, immune potentiation, and the prevention of malignant conversion (Le et al, J Biol. Chem. 2003 Jun. 6; 278(23):21136-45. Epub 2003 Mar. 27; U.S. Ser. No. 10/983,414)

Here we show that DIM compositions can be used as anti-angiogens, to effectively inhibit growth of actively proliferating vascular endothelial cells by retarding cell cycle progression. Furthermore, the antiproliferative effects of DIM compositions are relatively specific for active endothelial cells, and are observed at physiologically relevant concentrations. Although neoplastic cells readily acquire resistance to cytotoxic chemotherapy, genetically stable vascular endothelial cells have a more limited ability to develop drug resistance. Additionally, normal vascular endothelial cells turn over slowly, so an anti-angiogenic approach targeting fast proliferating vascular endothelial cells offers improved efficacy. Finally, the subject DIM compositions are of notably low toxicity and compatible with chronic delivery which is often necessary to arrest tumor development by inhibiting angiogenesis.

Relevant Literature

Aspects of this disclosure were published in Chang et al. Carcinogenesis. 2005 Apr.;26(4):771-8. Epub 2005 Jan. 20.

### SUMMARY OF THE INVENTION

The invention provides antiangiogenic compositions and methods of use. In one embodiment, methods involve providing an antiangiogen to a patient by (a) determining that the patient is. subject or predisposed to an androgen- and estrogen-independent hyperplasia, and in need of an antiangiogen; and (b) administering to the patient a predetermined amount of the antiangiogenic optionally substituted DIM, wherein the method may further comprise, after the adminstering step, specifically detecting a resultant inhibition of angiogenesis in the patient.

In another embodiment, the methods involve providing an antiangiogen to a patient determined to be subject to an androgen- and estrogen-independent hyperplasia, and in need of an antiangiogen by (a) administering to the patient a predetermined amount of the antiangiogenic optionally substituted DIM; and (b) specifically detecting a resultant inhibition of angiogenesis in the patient, wherein the method may further comprise, prior to the administrating step, determining that the patient is subject or predisposed to an androgen- and estrogen-independent hyperplasia, and in need of the antiangiogen.

In particular embodiments, the inhibition of angiogenesis is detected inferentially as an decrease in hyperplasia size.

The methods employ an antiangiogenic, optionally substituted 3,3′-diindolylmethane having formula I:

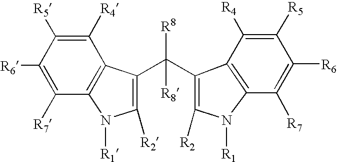

where R1, R2, R4, R5, R6, R7, R8, R1″, R2′, R4′, R5′, R6′, R7′ and R8′ individually and independently, are hydrogen or a substituent selected from the group consisting of a halogen, a hydroxyl, a nitro, —OR9, —CN, —NR9R10, —NR9R10R11⁺, —COR9, CF3, —S(O)nR9 (n=0-2), —SO2NR9R10, —CONR9R10, NR9COR10, —NR9C(O)NR10R11, —P(O)(OR9)n(n=1-2), optionally substituted alkyl, halovinyl, alkenyl, alkynyl, aryl, heteroalkyl, heteroaryl, or optionally substituted cycloalkyl or cycloakenyl, all of one to ten carbons and optionally containing 1-3 heteroatoms O or N, wherein R9, R10 and R11 are optionally substituted alkyl, alkenyl, alkynl, aryl, heteroalkyl, heteroaryl of one to ten carbons, and R8 and R8′ may further be O to create a ketone. In particular embodiments, the compound includes at least one such substituent, preferably at a position other than, or in addition to R1 and R1″, the linear or branched alkyl or alkoxy group is one to five carbons, and/or the halogen is selected from the group consisting of chlorine, iodine, bromine and fluorine. In particular embodiments, the indolyls are symmetrically substituted, wherein each indolyl is similarly mono-, di-, tri-, or para-substituted.

The invention also provides methods of using an antiangiogenic, optionally substituted 3,3′-diindolylmethane in conjunction with one or more other therapeutic agents, particularly different anticancer compounds, such as different antiangiogenic compounds, antimetabolites, etc., for complementary, additive, and/or synergistic efficacy. These methods may employ combination compositions, which may be in combination unit dosages, or separate compositions, which may be provided separately dosed in joint packaging.

The invention also provides kits comprising an antiangiogenic, optionally substituted 3,3′-diindolylmethane, and an instructional medium reciting a subject method. The recited antiangiogenic optionally substituted 3,3′-diindolylmethane may be present in premeasured, unit dosage, and may be combined in dosage or packaging with an additional therapeutic agent.

### DETAILED DESCRIPTION OF PARTICULAR EMBODIMENTS OF THE INVENTION

The following descriptions of particular embodiments and examples are offered by way of illustration and not by way of limitation. Unless contraindicated or noted otherwise, in these descriptions and throughout this specification, the terms “a” and “an” mean one or more, the term “or” means and/or.

The general methods deliver an antiangiogenic, optionally substituted DIM, to a host determined to be in need thereof. In one embodiment, methods involve providing an antiangiogen to a patient by (a) determining that the patient is subject or predisposed to an androgen- and estrogen-independent hyperplasia, and in need of an antiangiogen; and (b) administering to the patient a predetermined amount of the antiangiogenic optionally substituted DIM, wherein the method may further comprise, after the adminstering step, specifically detecting a resultant inhibition of angiogenesis in the patient.

In another embodiment, the methods involve providing an antiangiogen to a patient determined to be subject to an androgen- and estrogen-independent hyperplasia, and in need of an antiangiogen by (a) administering to the patient a predetermined amount of the antiangiogenic optionally substituted DIM; and (b) specifically detecting a resultant inhibition of angiogenesis in the patient, wherein the method may further comprise, prior to the administrating step, determining that the patient is subject or predisposed to an androgen- and estrogen-independent hyperplasia, and in need of the antiangiogen.

Target hyperplasias are hormone, particularly steroid sex-hormone (e.g. androgen- and estrogen) independent. Steroid sex hormone dependent hyperplasias include prostate and mammary cancers, and also papillomavirus-induced tumors, including tumors of the larynx and cervix wherein the pathogenesis is reportedly related to estrogen metabolism. Exemplary target hyperplasias include cancers of the lung and bronchus, colon and rectum, urinary tract and bladder, skin (e.g. melanoma), kidney and renal pelvis, pancreas, oral cavity and pharynx, brain, bone, and liver, as well as lymphomas (e.g. non-hodgkin lympohoma) and leukemias (e.g. myeloid and lymphocytic leukemias). In particular embodiments, the inhibition of angiogenesis is detected inferentially as a decrease in hyperplasia size.

Our methods employ an antiangiogenic, optionally substituted 3,3′-diindolylmethane having the structure of formula I, where R1, R2, R4, R5, R6, R7, R8, R1′, R2′, R4′, R5′, R6′, R7′ and R8′ individually and independently, are hydrogen or a substituent selected from the group consisting of a halogen, a hydroxyl, a nitro, —OR9, —CN, —NR9R10, —NR9R10R11⁺, —COR9, CF3, —S(O)nR9 (n=0-2), —SO2NR9R10, —CONR9R10, NR9COR10, —NR9C(O)NR10R11, —P(O)(OR9)nn=1-2), optionally substituted alkyl, halovinyl, alkenyl, alkynyl, aryl, heteroalkyl, heteroaryl, or optionally substituted cycloalkyl or cycloakenyl, all of one to ten carbons and optionally containing 1-3 heteroatoms O or N, wherein R9, R10 and R11 are optionally substituted alkyl, alkenyl, alkynl, aryl, heteroalkyl, heteroaryl of one to ten carbons, and R8 and R8′ may further be O to create a ketone. In particular embodiments, the compound includes at least one such substituent, preferably at a position other than, or in addition to R1 and R1″, the linear or branched alkyl or alkoxy group is one to five carbons, and/or the halogen is selected from the group consisting of chlorine, iodine, bromine and fluorine. Active DIM derivatives are readily obtained by SAR studies, e.g. Benabadji et al., Acta Pharmacol Sin. 2004 May; 25(5):666-71.

In particular embodiments, the optionally substituted DIMs are mono- and di-hydroxylated DIM derivatives at carbon positions 2, 4-7 and 2′, and 4′-7′, including each of [2, 4, 5, 6 or 7]-monohydroxy-DIM or [2′, 4′, 5′, 6′ or 7′]-monohydroxy-DIM (e.g. 2-hydroxy-DIM, 4-hydroxy-DIM etc.); and each of [2, 4, 5, 6 or 7], [2, 4, 5, 6 or 7]-dihydroxy-DIM, [2′, 4′, 5′, 6′ or 7′], [2′, 4′, 5′, 6′ or 7′]-dihydroxy-DIM, or [2, 4, 5, 6 or 7], [2′, 4′, 5′, 6′ or 7′]-dihydroxy-DIM (e.g. 2,4-dihydroxy-DIM, 2,5-dihydroxy-DIM etc, 2,2′-dihydroxy-DIM, 2,4′-dihydroxy-DIM etc.); particularly bilaterally symmetrical species, such as 2,2′-dihydroxy-DIM.

Antiangiogenic activity is readily confirmed with the various assays described below, including vascular endothelial cell proliferation assays, HUVEC capillary tube formation assays, and neoplastic xenograft assays. In particular, we devised an iterative, combinatorial synthetic scheme to generate a library of DIM derivatives for high-throughput screening of antiangiogenic activity. In an exemplary demonstration, we generated 451 DIM derivative structures in five synthetic rounds, summarized below:

Synthetic round 2: R2, 4, 5, 6 or 7, R2′, 4′, 5′, 6′ or 7′ di-F, —Cl, or —Br-3,3′-diindolylmethane

Synthetic round 5: R2, 4, 5, 6 or 7, R2′, 4′, 5′, 6′ or 7′ di-methyl-, ethyl-, propyl-, butyl-, or pentyl-3,3′-diindolylmethane

Synthetic round 6: R2, 4, 5, 6 or 7, R2′, 4′, 5′, 6′ or 7′ di-methoxy-, ethoxy-, propyloxy-, butyloxy-, or pentyloxy-3,3′-diindolylmethane

Synthetic round 9: R2, 4, 5, 6 or 7, R2′, 4′, 5′, 6′ or 7′ di-hydroxyl, amino-, aminomethyl-, sulfo-, or nitro-3,3′-diindolylmethane

Synthetic round 12: R2, 4, 5, 6, 7, R2′, 4′, 5′, 6′, 7′ deca-fluoro (perfluoro)-3,3′-diindolylmethane

Antiangiogenic effects of the DIM analogs are pre-screened with vascular endothelial cells proliferation assays. In our initial demonstrations, approximately 8×10⁴ HUVECs were seeded in 6-well plates and allowed to grow for 24 h. Cells were then treated by 0, 2, 5, 10, or 25 μM DIM candidate at 24, 48, and 72 h. DIM was dissolved in DMSO as 1000× stock solutions for each treatment. Cell numbers were assessed at various time points by trypsinization and counting with a Coulter Z1 cell counter, as described below in Example I below.

TABLE 1
Antiangiogenic substituted 3,3′-diindolylmethane compounds

| | |
|---|---|
| 5,5′-dichloro-diindolylmethane | 5,5′-dibromo-diindolylmethane |
| 5,5′-difluoro-diindolylmethane | perfluoro-3,3′-diindolylmethane |
| 5,5′-dimethyl-diindolylmethane | 5,5′-diethyl-diindolylmethane |
| 5,5′-dipropyl-diindolylmethane | 5,5′-dibutyl-diindolylmethane |
| 5,5′-dipentyl-diindolylmethane | 5,5′-dimethoxy-diindolylmethane |
| 5,5′-diethoxy-diindolylmethane | 5,5′-dipropyloxy-diindolylmethane |
| 5,5′-dibutyloxy-diindolylmethane | 5,5′-diamyloxy-diindolylmethane |
| N,N′- dimethyl-diindolylmethane | N,N′-diethyl-diindolylmethane |
| N,N′-dipropyl-diindolylmethane | N,N′-dibutyl-diindolylmethane |
| N,N′-dipentyl-diindolylmethane | 2,2′-dimethyl-diindolylmethane |
| 2,2′-diethyl-diindolylmethane | 2,2′-dipropyl-diindolylmethane |
| 2,2′-dibutyl-diindolylmethane | 2,2′-dipentyl-diindolylmethane |
| 2,2′-dihydroxy-diindolylmethane | 2-hydroxy-diindolylmethane |
| 2,4-dihydroxy-diindolylmethane | 2,2′-dihydroxy-diindolylmethane |
| 2,5-dihydroxy-diindolylmethane | 2,4′-dihydroxy-diindolylmethane |

In particular embodiments, the indolyl moieties are symmetrically substituted, wherein each moiety is similarly mono-, di-, tri-, etc. substituted. In other particular embodiments, R1, R2, R4, R6, R7, R8, R1″, R2′, R4′, R6′, R7′ and R8′ are hydrogen, and R5 and R5′ are a halogen selected from the group consisting of chlorine, iodine, bromine and fluorine. Additional DIM derivatives from which antiangiogenic compounds are identified as described herein include compounds wherein R1, R2, R4, R6, R7, R8, R1″, R2′, R4′, R6′, R7′ and R8′ are hydrogen, and R5 and R5′ are halogen. These include, but are not limited to 3,3′-diindolylmethane, 5,5′-dichloro-diindolylmethane; 5,5′-dibromo-diindolylmethane; and 5,5′-difluoro-diindolylmethane. Additional preferred such DIM derivatives include compounds wherein R1, R2, R4, R6, R7, R8, R1″, R2′, R4′, R6′, R7′ and R8′ are hydrogen, and R5 and R5′ are an alkyl or alkoxyl having from one to ten carbons, and most preferably one to five carbons. These include, but are not limited to 5,5′-dimethyl-diindolylmethane, 5,5′-diethyl-diindolylmethane, 5,5′-dipropyl-diindolylmethane, 5,5′-dibutyl-diindolylmethane and 5,5′-dipentyl-diindolylmethane. These also include, but are not limited to, 5,5′-dimethoxy-diindolylmethane, 5,5′-diethoxy-diindolylmethane, 5,5′-dipropyloxy-diindolylmethane, 5,5′-dibutyloxy-diindolylmethane, and 5,5′-diamyloxy-diindolylmethane.

Additional preferred such DIM derivatives include compounds wherein R2, R4, R5, R6, R7, R8, R2′, R4′, R5′, R6′, R7′ and R8′ are hydrogen, and R1 and R1′ are an alkyl or alkoxyl having from one to ten carbons, and most preferably one to five carbons. Such useful derivatives include, but are not limited to, N,N′-dimethyl-diindolylmethane, N,N′-diethyl-diindolylmethane, N,N′-dipropyl-diindolylmethane, N,N′-dibutyl-diindolylmethane, and N,N′-dipentyl-diindolylmethane. In yet another preferred embodiment, R1, R4, R5, R6, R7, R8, R1″, R4′, R5′, R6′, R7′ and R8′ are hydrogen, and R2 and R2′ are alkyl of one to ten carbons, and most preferably one to five carbons. Such compounds include, but are not limited to, 2,2′-dimethyl-diindolylmethane, 2,2′-diethyl-diindolylmethane, 2,2′-dipropyl-diindolylmethane, 2,2′-dibutyl-diindolylmethane, and 2,2′-dipentyl-diindolylmethane. In another embodiment, R1, R2, R4, R6, R7, R8, R1″, R2′, R4′, R6′, R7′, and R8′ are hydrogen, and R5 and R5′ are nitro.

Substituted DIM analogs are readily prepared by condensation of formaldehyde with commercially available substituted indoles. Precursor compounds can be synthesized by dimethylformamide condensation of a suitable substituted indole to form a substituted indole-3-aldehyde. Suitable substituted indoles include indoles having substituents at R1, R2, R4, R5, R6 and R7 positions. These include, but are not limited to 5-methoxy, 5-chloro, 5-bromo, 5-fluoro, 5′-methyl, 5-nitro, n-methyl and 2-methyl indoles. The substituted indole 3-aldehyde product is treated with a suitable alcohol such as methanol and solid sodium borohydride to reduce the aldehyde moiety to give substituted 13Cs. Substituted DIMs are prepared by condensing the substituted indole-3-carbinol products. This may be achieved, for example, by treatment with a phosphate buffer having a pH of around 5.5.

The subject compositions may be administered along with a pharmaceutical carrier and/or diluent. Examples of pharmaceutical carriers or diluents useful in the present invention include any physiological buffered medium, i.e., about pH 7.0 to 7.4 comprising a suitable water soluble organic carrier. Suitable water-soluble organic carriers include, but are not limited to corn oil, dimethylsulfoxide, gelatin capsules, etc. The antiangiogenic compositions of the present invention may also be administered in combination with other agents, for example, in association with other chemotherapeutic or antiangiogenic drugs or therapeutic agents. These methods may employ combination compositions, which may be in combination unit dosages, or separate compositions, which may be provided separately dosed in joint packaging. In particular embodiments, the invention provides methods of using the subject antiangiogenic, optionally substituted 3,3′-diindolylmethane in conjunction with one or more other therapeutic agents, particularly different antiangiogenic compounds, for complementary, additive, and/or synergistic efficacy.

The compositions of the present invention may be administered orally, intravenously, intranasally, rectally, or by any means that delivers an effective amount of the active agent(s) to the tissue or site to be treated. Suitable dosages are those that achieve the desired endpoint. It will be appreciated that different dosages may be required for treating different disorders. An effective amount of an agent is that amount which causes a significant decrease in the targeted pathology, or progress of the pathology, or which delays the onset or reduces the likelihood of pathology in predisposed hosts. For example, the effective amount may decrease hyperplasia size or sample cell count, growth rate, associated pathology, etc.

The administered antiangiogenic, optionally substituted DIM may be advantageously complexed or coadministered with one or more functional moiety to provide enhanced update, bioavailability, stability, half-life, etc., or to reduce toxicity, etc. However, the compositions nevertheless comprise the recited antiangiogenic, optionally substituted DIM, whether in isolated, complexed, or a pro-drug form.

Those having ordinary skill in the art will be able to empirically ascertain the most effective dose and times for administering the agents of the present invention, considering route of delivery, metabolism of the compound, and other pharmacokinetic parameters such as volume of distribution, clearance, age of the subject, etc.

The invention also provides kits specifically tailored to practicing the subject methods, including kits comprising an antiangiogenic, optionally substituted 3,3′-diindolylmethane, and an associated, such as copackaged, instructional medium describing or reciting a subject method. The recited antiangiogenic, optionally substituted 3,3′-diindolylmethane may be present in premeasured, unit dosage, and may be combined in dosage or packaging with an additional therapeutic agent, particularly a different antiangiogen or other antineoplastic agent, such as endostatin, VEGF, avastatin, enzastaurin, resveratrol, thalidomide, etc.

The invention also provides business methods specifically tailored to practicing the subject methods. For example, in one embodiment, the business methods comprise selling, contracting, or licensing a subject, antiangiogenic, optionally substituted 3,3′-diindolylmethane-based method or composition.

The present invention is exemplified in terms of in vitro and in vivo activity against growth of various hyperplasias and neoplastic cell lines. The test cell lines employed in the in vitro assays are well recognized and accepted as models for antiangiogenesis. The mouse experimental in vivo assays are also well recognized and accepted as predictive of in vivo activity in other animals such as, but not limited to, humans.

### Exemplary Empirical Protocols

I. DIM strongly inhibits proliferation, migration, invasion, and capillary tube formation in cultured HUVECs. This example also shows that DIM strongly inhibits vascularization in a Matrigel plug assay, and tumorigenesis in a human tumor cell xenograft assay.

Cell proliferation assay. To determine endothelial cell proliferation, approximately 8×10⁴ HUVECs were seeded in 6-well plates and allowed to grow for 24 h. Cells were then treated by 0, 2, 5, 10, or 25 μM DIM at 24, 48, and 72 h. DIM was dissolved in DMSO as 1000× stock solutions for each treatment. Cell numbers were assessed at various time points by trypsinization and counting with a Coulter Z1 cell counter.

Cell invasion assay. Invasion assays were carried out using modified transwell Boyden chamber system. Chambers were assembled using 8 μm-pore BD Falcon transwell inserts as upper chambers, and the 12-well plates as lower chambers. Cell culture inserts were coated with 10 μl/insert Matrigel. Coated inserts were left to dry overnight in a laminar flow fume hood and then rehydrated with DMEM supplemented with 0.1% BSA for 1 h at 37° C. Rehydrated Matrigel wells were washed with the same medium. HUVECs were harvested by trypsin/EDTA and resuspended in 0.1% BSA/DMEM. Medium containing 5% FBS was applied to the lower chamber as chemoattractant, then cells were seeded at 1.5×10⁵ cells/insert in the presence of 0, 5, 10 or 25 μM DIM and incubated for 2 h at 37° C. and 5% CO₂. At the end of the incubation, the cells in the upper chamber were removed with cotton swabs and cells that traversed the Matrigel to the lower surface of the insert were fixed with 10% formalin/PBS and stained with crystal violet in 10% formalin/PBS. Cells that migrated to the lower surface of the insert were counted under the light microscope at a magnification of 40×. Each treatment was in triplicate. The quantification of the invasive cells in the presence of DIM was expressed as the percentage of the quantity of invasive cells under control conditions (DMSO).

Wound migration assay. Migration was assessed using an in vitro wound assay. One hundred thousands cells were seeded into two 12-well cell culture plates and cultured in EGM-MV BulletKit to confluency. A scrape was made in the center of the cell monolayers with a sterile pipette tip to create a gap of constant width. Cellular debris was removed by gently washing with PBS. The initial images of the wounds were captured under phase contrast microscopy and the wounded monolayers were incubated further for 18 h in fresh EGM-MV BulletKit medium in the presence of DMSO or DIM of various concentrations (5, 10, or 25 μM). Pictures were taken with a Nikon Coolpix990 digital camera connected to the Nikon Eclipse TS 100 microscope at a 100× magnification. To quantify the migration, photographs of the initial wounded monolayers were compared with the corresponding images of cells at the end of the incubation. Artificial lines fitting the cutting edges were drawn on pictures of the original wounds and overlaid on images of cultures after incubation. Cells that migrated across the lines were counted. All quantification were done on full-size images with the weight of artificial lines negligible compared to the size of the cell body. At least 5 fields from each triplicate treatment were counted.

Tube formation assay. Endothelial cells plated onto a gel of basement membrane protein rapidly organize into multicellular tube-like structures [12; 13]. Twelve-well cell culture plates coated with Matrigel were ordered from BD-Discovery Labware and assay was performed according to the manufacturer's instruction. Briefly, Matrigel-coated 12-well plates were allowed to solidify at 37° C. for 1 h. Endothelial cells pre-treated with 0, 10, or 25 μM DIM for 24 h were trypsinized and 6×10⁴ cells were added per well in 1 mL medium. Cell viability was determined by trypan blue exclusion stain before seeding. DIM treatments continued for the rest of the assay. Tube formation was observed periodically over time under a phase contrast microscope, and representative pictures were taken at 3 h and 24 h.

Matrigel plug angiogenesis assay. In vivo angiogenesis assays were performed on female C57BL/6 mice as described before [14]. Mice were acclimated to semi-purified phytoestrogen-free AIN-76 ad libitum for 7 days prior to the study and randomly grouped (5/group). DMSO vehicle or DIM at 5 mg/kg was injected s.c. five days before Matrigel inoculation. Heparin (64 Unit/mL) and aFGF (100 ng/mL) were gently mixed with cold liquid Matrigel at 4° C. The Matrigel solution (0.3 ml) was injected into mice s.c. in the bilateral flanks. Animals were treated with DMSO control or DIM for 2 more weeks and terminated by CO₂ inhalation. The gels were surgically removed and vascularization of Matrigel plugs was quantified by measuring hemoglobin content using Drabkin Reagent kit 525.

DIM inhibits proliferation, invasion, migration and tube formation of HUVECs. We conducted a series of assays in primary cell culture to determine whether DIM exhibits antiangiogenic potential. First, DIM's effect on proliferation of human vascular endothelial cell (HUVECs) was examined. HUVECs were treated with 0, 2, 5, 10, or 25 μM DIM for 24 to 72 h. DIM inhibited HUVECs proliferation in a concentration- and time-dependent manner, with up to 70% inhibition of proliferation at 72 h treatment with 25 μM DIM. Significant inhibition of proliferation occurred in cells treated with only 5 μM DIM at 72 h. Cell survival and viability, as was determined by trypan blue exclusion, were not obviously affected by the conditions of this assay. These results indicate DIM acts as an angiogenesis inhibitor by directly reducing vascular endothelial cells proliferation.

We next conducted a cell invasion assay to examine the effect of DIM on HUVEC movement through a simulated extracellular matrix. In a modified Boyden chamber assay, the transwell inserts were coated with a thin layer of Matrigel and inserted into wells containing 5% FBS medium as a chemoattractant. As a negative control, serum-free medium containing 0.1% BSA was used in the lower chamber wells. Cells which transversed the Matrigel and migrated to the lower part of the insert were photographed and quantified. The results showed that no cell invasion was observed with serum-free medium in the lower chamber (negative control). In response to 5% FBS, however, HUVECs traversed the Matrigel and migrated to the lower chamber. The invasion of HUVECs was decreased by DIM in a concentration-dependent manner, in which DIM at 25 μM decreased cell invasion to 52% of the control. The trend of decreased cell invasion was seen at 5 μM DIM and a more significant effect was seen at 10 μM exposure.

A complementary wound migration assay was also conducted as described above to determine whether DIM directly affects cell migration. For this assay, confluent cultures of HUVECs were wounded and then incubated for 18 h in fresh complete medium. To quantify the migration, artificial lines fitting the cutting edges were drawn on pictures of the original wounded cells and overlaid on images of cells after incubation. Cells that migrated across the lines were counted. Images of the migration assay including an example of the artificial lines drawn for quantification show that the wound was largely closed by cells migrating from both edges of the wound after 8 h in the DMSO control. However, DIM inhibited this process in a concentration dependent manner. HUVECs treated with DIM showed a dramatic decrease in migration of cells across the wound, with 25 μM DIM decreasing the number to only 40% of control.

In a further assay of angiogenesis, we examined the effect of DIM on HUVEC tube formation in culture. Capillary formation on Matrigel is a process that requires cell-matrix interaction. For the assay, HUVECs were pre-treated without or with 10 or 25 μM of DIM for 24 h and then seeded on a thin layer of Matrigel. Capillary tube structure formation was obvious at 3 h of incubation and was almost completed at 24 h in the control medium. However, DIM-treated HUVECs showed decreased ability to extend and differentiate into tube-like structures with effects obvious following 3-h incubation with 10 μM DIM. Taken together, our results indicate that DIM acted directly on cultured endothelial cells to inhibit processes of proliferation, migration and tube formation that are important markers of potential antiangiogenic activity, in vivo.

DIM induces a G1 cell cycle arrest in HUVECs. To further characterize the antiproliferative activity of DIM in HUVECs, we examined the effects of this indole on cell cycle regulation. For the cell cycle studies, HUVECs treated with vehicle control or 25 μM DIM for 24 h were analyzed by flow cytometry as described above. Histograms of cell cycle distribution in control and 25 μM DIM treated HUVECs show DIM treatment increased the cell population in G1 phase in a concentration-dependent manner, which became evident following 24 h treatment of an asynchronous growing cell population with 5 μM DIM. Conversely, the cell population in S phase significantly deceased. For example, 25 μM DIM treatment increased the proportion of cells in the G1 phase from 71.8±2.5% to 85.7±2.9%, and decreased the proportion of S phase cells from 15.2±1.3 to 2.4±0.9% (P<0.01), clearly indicating a G1 block in cell cycle progression.

DIM down-regulates CDK2 and CDK6, and up-regulates CDK inhibitor p27^{Kip1} in HUVECs. Our observation that DIM induces a G1 block in cell cycle progression suggested that DIM might selectively regulate the activities of G1 cell cycle regulating components. To examine this possibility, the expressions of G1 cell cycle components were investigated by Western blot analysis. Our results indicate that DIM treatment reduced the expression of CDK2 and CDK6 protein and strongly increased the expression of a cell cycle inhibitor (CKI) p27^{Kip1}. Levels of CDK4, cyclin E and p21^{Waf1/CiP1}, however, were not significantly affected by DIM treatment. DIM (25 μM) treatment significantly decreased CDK2 expression to 35% and CDK6 to 40% of the control. In contrast, DIM significantly increased CDK inhibitor p27^{Kip1} to nearly 3.5 fold of the control with 25 μM DIM administration. These data indicate that DIM induces a G1 cell cycle arrest in HUVECs, which is accompanied by down-regulation of expression of CDK2 and CDK6 G1-related kinases and up-regulation of expression of the CDK inhibitor, p27^{Kip1}.

DIM reduces in vivo angiogenesis. Using a rodent Matrigel plug angiogenesis assay, we next investigated whether DIM affects neovascularization in vivo. Matrigel is a urea extract of Engelbreth-Holm-Swarm (EHS) tumor, which contains laminin, collagen IV, heparan sulfate proteoglycan, and several growth factors, all of which are present in the authentic basement membrane of solid tumors. When injected subcutaneously into rodents, Matrigel forms a solid “plug” beneath the skin. The hemoglobin content in the Matrigel parallels blood vessels development in the gel, thereby allowing quantitation [14]. Matrigel (0.3 mL) containing 64 unit/mL heparin and 100 ng/mL aFGF were injected s.c. into the bilateral flanks of C57/BL mice. DMSO vehicle or 5 mg/kg of DIM was injected s.c. starting five days before Matrigel inoculation and continuing for the remaining 2 weeks of the experiment. The result showed that DIM treatment reduced neovascularization up to 76% compared to controls, as indicated by significantly lower hemoglobin content. Thus, DIM exhibits direct inhibitory activity on angiogenesis.

II. Antiangiogenic DIM inhibits human breast carcinoma in xenograft model. Twenty female athymic (nu/nu) mice were acclimated to semi-purified phytoestrogen-free AIN-76 ad libitum for 7 days prior to the study. They were implanted with a 60-d release 0.72 mg estradiol pellet in the subscapular region. Mice were then inoculated s.c. in the bilateral flanks with 0.1 mL Matrigel containing 3×10⁶ MCF-7 human breast cancer cells and randomly grouped (10/group) to receive s.c. injections of either 5 mg/kg DIM or DMSO in a PBS vehicle, five times weekly. Feed intake, body weight were measured weekly and palpable tumor diameter were measured twice per week. Tumor volumes were calculated as: (π/6)×[length(mm)×width²(mm)] [15]. The experiment was terminated at 34 days. All animals were killed by CO₂ asphyxiation.

DIM inhibits the growth of transplanted MCF-7 human breast carcinoma cells. Since we observed an inhibition of in vivo angiogenesis by DIM in the Matrigel plug assay, we determined whether DIM could inhibit the growth of transplanted breast carcinoma cells in female athymic (nu/nu) mice. Mice were injected s.c. with MCF-7 human breast cancer cells in the bilateral flanks. Mice were randomly assigned (10 mice/group) to receive s.c. injection of either DIM at 5 mg/kg dose or vehicle control DMSO in PBS, five times weekly. Feed intake, body weight and palpable tumor diameter were measured twice per week. Feed intake (22.2±1.3 g/wk vs. 22.9±1.0 g/wk) and body weight gain (2.3±0.2 g vs. 2.4±0.2 g) were not altered by DIM administration. Furthermore, relative organ weights were not significantly affected by DIM treatment, indicating that the dose of DIM used in this study did not cause overt toxicity. Tumor volumes were calculated as: (π/6)×[length(mm)×width²(mm²)]. DIM treatment reduced tumor growth by 40% (P=0.22) after 3 weeks and by 64% (P<0.05) at termination of the study at day 34. Final average tumor volume was significantly lower in the DIM group (1125±434 mm³) compared to the control group (3121±554.5 mm³). These results show that DIM can strongly decrease development of estrogen-dependant human breast cell tumors in a rodent xenograft model.

III. Antiangiogenic DIM compositions inhibit human pancreatic carcinoma xenografts in nude mice.

Our protocol for investigating the anti-tumor effects of antiangiogenic, optionally substituted DIM therapy on human pancreatic carcinoma xenografts was adapted from Jia et al., World J Gastroenterol. 2005 Jan. 21; 11(3):447-50. Briefly, a surgical orthotopic implantation (SOI) model is established by suturing small pieces of SW1990 pancreatic carcinoma into the tail of pancreas in nude male mice. Mice then receive graduated dosages (1 or 10 mg/kg) of the antiangiogenic DIM composition of Table 1 IP on d 0, 3, 6 and 9 after transplantation. Animals are killed 8 wk after transplantation, and transplanted tumors, liver, lymph node and peritoneum removed. Weight of transplanted tumors, the T/C rate (the rate of mean treated tumor weight to mean control tumor weight), change of body weight, metastasis rate, and 9-wk survival rate are investigated. Tumor samples are taken from the control and treatment groups. PCNA index (PI) and microvessel density (MVD) are investigated by immunohistochemical staining for PCNA and factor VIII, respectively. Results demonstrate a significant inhibitory effect on primary tumor growth of pancreatic carcinoma in the DIM treatment groups. Antiangiogenic therapy shows significant anti-tumor and anti-metastatic effects.

IV. Antiangiogenic DIM compositions synergize with gemcitabine to inhibit human pancreatic carcinoma xenografts in nude mice.

Our protocol for investigating the anti-tumor effects of antiangiogenic, optionally substituted DIM therapy in combination with antimetabolite therapy on human pancreatic carcinoma xenografts was adapted from Jia et al., World J Gastroenterol. 2005 Jan. 21; 11(3):447-50. Briefly, a surgical orthotopic implantation (SOI) model is established by suturing small pieces of SW1990 pancreatic carcinoma into the tail of pancreas in nude male mice. Mice then receive graduated dosages (1 or 10 mg/kg) of the antiangiogenic DIM composition of Table 1, with and without coadministration of gemcitabine at either low (50 mg/kg) or high (100 mg/kg) dosage, IP on d 0, 3, 6 and 9 after transplantation. Animals are killed 8 wk after transplantation, and transplanted tumors, liver, lymph node and peritoneum removed. Weight of transplanted tumors, the T/C rate (the rate of mean treated tumor weight to mean control tumor weight), change of body weight, metastasis rate, and 9-wk survival rate are investigated. Tumor samples are taken from the control and treatment groups. PCNA index (PI) and microvessel density (MVD) are investigated by immunohistochemical staining for PCNA and factor VIII, respectively. Results again demonstrate a significant inhibitory effect on primary tumor growth of pancreatic carcinoma in the DIM treatment groups, and antiangiogenic therapy shows significant anti-tumor and anti-metastatic effects. However, these results also demonstrate a reduced the dosage requirement for the coadministered cytotoxic antimetabolite.

V. Antiangiogenic DIM compositions inhibit human pancreatic carcinoma xenografts in nude mice.

Our protocol for investigating the anticancer activity of antiangiogenic, optionally substituted DIM on implanted human primary gastric carcinoma cells in nude mice was adapted from Zhou et al., World J Gastroenterol. 2005 Jan. 14; 11(2):280-4. Briefly, a transplanted tumor model is established by injecting human primary gastric cancer cells into subcutaneous tissue of nude mice. DIM compositions of Table 1 at 1 or 10 mg/kg are directly injected beside tumor body 6 times at an interval of 2 d. Then changes of tumor volume are measured continuously and tumor inhibition rate of each group calculated. Results demonstrate that the DIM compositions significantly inhibit carcinoma growth when proximally injected.

VI. Antiangiogenic DIM compositions inhibit human pancreatic carcinoma xenografts in nude mice.

Our protocol for investigating the effects of our DIM compositions on angiogenesis and tumor growth and metastasis of human hepatocellular carcinoma in nude mice was adapted from Zhang et al., World J Gastroenterol. 2005 Jan. 14; 11(2):216-20. Briefly, nude mice are randomly divided into therapy and control groups, 12 mice in each group. DIM compositions of Table 1 dissolved in 0.5% sodium carboxylmethyl cellulose (CMC) suspension are administered intraperitoneally once a day at doses of 1 or 10 mg/kg in the therapy groups, and an equivalent volume of 0.5% CMC in control group. Mice are sacrificed on the 30th day, and tumor size and weight and metastases in liver and lungs measured. Results confirm that our DIM compositions can significantly inhibit angiogenesis and metastasis of hepatocellular carcinoma.

### REFERENCES

1. Folkman, J. (1995) Angiogenesis in cancer, vascular, rheumatoid and other disease. Nat. Med., 1, 27-31.
2. Folkman, J. (1995) Seminars in Medicine of the Beth Israel Hospital, Boston. Clinical applications of research on angiogenesis. N. Engl. J. Med., 333, 1757-1763.
3. Folkman, J., et al. (1971) Tumor angiogenesis—Therapeutic implications. N. Engl. J. Med., 285, 1182-1186.
4. Folkman, J. and Shing, Y. (1992) Angiogenesis. J. Biol. Chem., 267, 10931-10934.
5. Pili, R., et al. (1995) Cancer Res., 55, 2920-2926.
6. Folkman, J. (1975) Ann. Intern. Med., 82, 96-100.
7. McDougal, A., et al. (2000). Cancer Lett., 151, 169-179.
8. Bjeldanes, L. F., et al. (1991) Proc. Natl. Acad. Sci. U.S.A, 88, 9543-9547.
9. Bradfield and Bjeldanes (1987) J. Toxicol. Environ. Health, 21, 311-323.
10. Chen et al. (1998) Aryl hydrocarbon receptor-mediated antiestrogenic and antitumorigenic activity of diindolylmethane. Carcinogenesis, 19, 1631-1639.
11. Grose, K. R. and Bjeldanes, L. F. (1992) Oligomerization of indole-3-carbinol in aqueous acid. Chem. Res. Toxicol., 5, 188-193.
12. Grant, et al. (1989) Two different laminin domains mediate the differentiation of human endothelial cells into capillary-like structures in vitro. Cell, 58, 933-943.
13. Kubota, Y., Kleinman, H. K., Martin, G. R., and Lawley, T. J. (1988) Role of laminin and basement membrane in the morphological differentiation of human endothelial cells into capillary-like structures. J. Cell Biol., 107, 1589-1598.
14. Passaniti, A., et al. (1992) A simple, quantitative method for assessing angiogenesis and antiangiogenic agents using reconstituted basement membrane, heparin, and fibroblast growth factor. Lab Invest, 67, 519-528.
15. Tomayko, M. M. and Reynolds, C. P. (1989) Determination of subcutaneous tumor size in athymic (nude) mice. Cancer Chemother. Pharmacol., 24, 148-154.

The foregoing descriptions of particular embodiments and examples are offered by way of illustration and not by way of limitation. All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of providing an antiangiogen to a patient, comprising the steps of:
determining that the patient is subject or predisposed to an androgen- and estrogen-independent hyperplasia, and in need of an antiangiogen; and
administering to the patient a predetermined amount of an antiangiogenic optionally substituted 3,3′-diindolylmethane.

2. A method of providing an antiangiogen to a patient determined to be subject to an androgen- and estrogen-independent hyperplasia, and in need of an antiangiogen, comprising the steps of:
administering to the patient a predetermined amount of an antiangiogenic optionally substituted 3,3′-diindolylmethane; and
specifically detecting a resultant inhibition of angiogenesis in the patient.

3. The method of claim 1, wherein the method further comprises, after the adminstering step, specifically detecting a resultant inhibition of angiogenesis in the patient.

4. The method of claim 2 wherein the method further comprises, prior to the administrating step, determining that the patient is subject or predisposed to an androgen- and estrogen-independent hyperplasia, and in need of the antiangiogen.

5. The method of claim 2 wherein the inhibition of angiogenesis is detected inferentially as a decrease in hyperplasia size.

6. The method of claim 1 wherein the administering step is performed by oral or intravenous administration.

7. The method of claim 1 wherein the optionally substituted 3,3′-diindolylmethane has the formula: 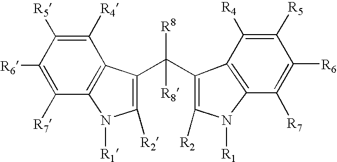
where R1, R2, R4, R5, R6, R7, R8, R1″, R2′, R4′, R5′, R6′, R7′ and R8′ individually and independently, are hydrogen or a substituent selected from the group consisting of a halogen, a hydroxyl, a nitro, —OR9, —CN, —NR9R10, —NR9R10R11⁺, —COR9, CF3, —S(O)nR9 (n=0-2), —SO2NR9R10, —CONR9R10, NR9COR10, —NR9C(O)NR10R11, —P(O)(OR9)n(n=1-2), optionally substituted alkyl, halovinyl, alkenyl, alkynyl, aryl, heteroalkyl, heteroaryl, or optionally substituted cycloalkyl or cycloakenyl, all of one to ten carbons and optionally containing 1-3 heteroatoms O or N, wherein R9, R10 and R11 are optionally substituted alkyl, alkenyl, alkynl, aryl, heteroalkyl, heteroaryl of one to ten carbons, and R8 and R8′ may further be O to create a ketone.

8. The method of claim 7 wherein R1, R2, R4, R5, R6, R7, R8, R1″, R2′, R4′, R5′, R6′, R7′ and R8′ include a substituent selected from the group consisting of a halogen, a hydroxyl, a linear or branched alkyl or alkoxy group of one to ten carbons, and a nitro group.

9. The method of claim 8 wherein the linear or branched alkyl or alkoxy group is one to five carbons.

10. The method of claim 8 wherein the halogen is selected from the group consisting of chlorine, iodine, bromine and fluorine.

11. The method of claim 8, wherein R1, R2, R4, R6, R7, R8, R1″, R2′, R4′, R6′, R7′ and R8′ are hydrogen, and R5 and R5′ are a halogen.

12. The method of claim 8, wherein R2, R4, R5, R6, R7, R8, R2′, R4′, R5′, R6′, R7′ and R8′ are hydrogen, and R1 and R1′ are an alkyl or alkoxyl having from one to ten carbons.

13. The method of claim 8, wherein R1, R4, R5, R6, R7, R8, R1″, R4′, R5′, R6′, R7′ and R8′ are hydrogen, and R2 and R2′ are an alkyl of one to ten carbons.

14. The method of claim 8, wherein R1, R2, R4, R6, R7, R8, R1″, R2′, R4′, R6′, R7′ and R8′ are hydrogen, and R5 and R5′ are nitro.

15. The method of claim 1 wherein the optionally substituted 3,3′-diindolylmethane is 3,3′-diindolylmethane.

16. The method of claim 2 wherein the optionally substituted 3,3′-diindolylmethane is 3,3′-diindolylmethane.

17. The method of claim 1 wherein the optionally substituted 3,3′-diindolylmethane is perfluoro-3,3′-diindolylmethane.

18. The method of claim 2 wherein the optionally substituted 3,3′-diindolylmethane is perfluoro-3,3′-diindolylmethane.

19. The method of claim 1 wherein the optionally substituted 3,3′-diindolylmethane is 2,2′-dihydroxy-diindolylmethane.

20. The method of claim 2 wherein the optionally substituted 3,3′-diindolylmethane is 2,2′-dihydroxy-diindolylmethane.
